(19) United States
(12) Patent Application Publication
(10) Pub. No.: US 2020/0023067 A1
(43) Pub. Date: Jan. 23, 2020

(54) DUAL-ACTION ELAFIBRANOR METFORMIN SALT FOR TREATING OBESITY ASSOCIATED WITH NON-ALCOHOLIC STEATOHEPATITIS (NASH) AND HYPERTRIGLYCERIDAEMIA

(71) Applicant: NASHPHARM, Villeneuve Loubet (FR)

(72) Inventors: Claude LARUELLE, Villeneuve Loubet (FR); Ludovic BONNAFOUS, Mouans Sartoux (FR)

(21) Appl. No.: 16/338,189

(22) PCT Filed: Sep. 28, 2017
(86) PCT No.: PCT/EP2017/074703

§ 371 Date: May 3, 2019

(30) Foreign Application Priority Data
Sep. 30, 2016 (FR) 1659435

(51) Int. Cl. A61K 47/55; A61K 31/155; A61K 31/19; A61K 9/00

(52) U.S. Cl. CPC A61K 47/55; A61K 31/155; A61K 9/0019; A61K 9/0053; A61K 31/19

(57) ABSTRACT

Drugs derived from elafibranor. One or more embodiments relate more particularly to a composition comprising at least one active principle, wherein the at least one active principle comprises an elafibranor metformin salt. One or more embodiments also relate to a derivative of elafibranor having dual action for treating obesity associated with non-alcoholic steatohepatitis (NASH) and hypertriglyceridaemia.

### TECHNICAL FIELD

The present invention relates to drugs derived from elafibranor.

The present invention relates more particularly to a derivative of elafibranor having dual action for treating obesity associated with non-alcoholic steatohepatitis (NASH) and hypertriglyceridaemia. This involves a novel product, elafibranor metformin salt (GFT505), a method for preparing said novel product, and pharmaceutical compositions containing said novel product as an active principle.

The invention also relates to pharmaceutical formulations in various forms of administration by enteral or parenteral route for treating or preventing illnesses resulting from the metabolic syndrome such as obesity, excessive weight, diabetes, insulin resistance, dyslipidaemia, hepatic diseases including steatosis, fibrosis or cirrhosis, and cardiovascular illnesses that result therefrom. The invention relates more precisely to pharmaceutical compositions for treating or preventing obesity associated with non-alcoholic steatohepatitis (NASH).

### PRIOR ART

Obesity and weight problems are a major problem in western countries. In 2012 in France there were 24.6 million persons overweight, that is to say one third of the population. Half have a weight problem. Obesity appears to be the cause of 13% of deaths in Europe. There is therefore a vital need for treating these illnesses with the search for more effective active molecules.

The elafibranor described in this invention refers to the experimental module from the company Genfit. It may appear cited in its code name GFT505 or GFT-505, developed initially for treating metabolic illnesses including diabetes, insulin resistance and dyslipidaemia. Its current therapeutic target is the treatment of liver diseases, in particular non-alcoholic steatohepatitis (NASH).

Its chemical name is 2-[2,6 dimethyl-4-[3-[4-(methylthio)phenyl]-3-oxo-1(E)-propenyl]phenoxyl]-2-methylpropanoic acid, of chemical formula C₂₂H₂₄O₄S and with a molecular weight of 384.489 g/mol. Its chemical structure of formula I is given in FIG. 1.

The Genfit patent EP 1525177 B1 describes the use and preparation of molecules of the 1,3-diphenylprop-2-en-1-one family. Elafibranor, referred to as compound 29 in this document, is identified by an MNR spectrum (1H DMSO) with the following characteristics (δ ppm): 1.39 (s, 6H), 2.22 (s, 6H), 2.57 (s, 3H), 7.40 (d, J=8.55 Hz, 2H), 7.57 (s, 2H), 7.62 (d, J=15.5 Hz, 1H), 7.83 (d, J=15.5 Hz, 1H), 8.10 (d, J=8.55 Hz, 2H), 12.97 (s, 1H) SM (ES-MS): 383.3 (M−1).

No publically available document gives any more information about the physical and chemical identification of this molecule, which comprises the same phenoxylated propanoic acid group as the molecules in the fibrate family (FIG. 2).

The molecules belonging to the fibrate family are known for their low solubility in water and for lowering the plasmatic concentration of triglycerides and that of cholesterol of very low density lipoproteins.

Fibrates are known for activating nuclear receptors known as PPAR (peroxisome proliferator activating receptors), in particular the alpha isoforms, which regulate the transcription of the genes involved in the metabolism of lipoproteins rich in triglycerides and HDLs (“good cholesterol”).

The other information available on the elafibranor molecule concerns mainly the preclinical, clinical and toxicological properties of this molecule. Like fibrates, elafibranor is identified as being a coactivator of the PPAR α/δ nuclear receptors. Clinical tests show a very good profile of tolerance of this molecule, reinforced in particular by toxicological studies at high doses in animals, including carcinogenicity studies.

Elafibranor (GFT05) also has beneficial effects on non-alcoholic steatohepatitis NASH with an improvement in the biochemical markers of hepatic malfunctioning, such as the hepatic enzymes: ALAT, ASAT, γGT, and ALP7.

Currently there are no thorough descriptions of the physical and chemical data of elafibranor, whether it be the chemical molecule alone or as an active molecule in a pharmaceutical composition. No physiologically acceptable salt is described in patents or scientific publications.

Elafibranor (GFT505) has been described since 2003 in several patents of the company Genfit, which cover any therapeutic application and, since 2009, on a novel specific therapeutic application, in particular for treating non-alcoholic steatohepatitis NASH.

The patents EP1525177 and U.S. Pat. No. 7,943,661 relate to a novel family of derivatives of chalcones. They describe the method for preparing and using substituted derivatives of 1,3-diphenylprop-2-en-1-one of formula II below (FIG. 4), of which the elafibranor molecule forms part (compound 29 described in the description, claim 25), for any therapeutic application, without limitation to a precise illness.

The second family of patents EP2504005, U.S. Pat. Nos. 8,772,342 and 9,221,751 relate to compounds for use in a method for treating a liver disease chosen from the group consisting of hepatic fibrosis or hepatic steatosis.

In particular, claim 7 relates to the elafibranor molecule for use in the treatment of hepatic fibrosis or hepatic steatosis. Claims 9 and 10 relate to a pharmaceutical composition comprising a compound of the following formula III (FIG. 5) in a method for treating a liver disease chosen from the group consisting of hepatic fibrosis or hepatic steatosis.

The patent EP2504005B1 was the subject of a divisional application EP2641596A1 concerning the compounds claimed in the patent EP2504005B1, but used this time solely in the specific context of the illnesses: cirrhosis of the liver, alcohol-related illnesses and immune mediated liver diseases.

Other patents dealing with elafibranor should be noted. The patent U.S. Pat. No. 7,566,737B relates to a pharmaceutical composition comprising an association between a substituted derivative of 1,3-diphenylprop-2-en-1-one of formula II, including the elafibranor molecule, and another ingredient having therapeutic activity.

The patent U.S. Pat. No. 8,895,619 B relates to a method for treating hepatic fibrosis by the administration of the elafibranor molecule (claims 1-7, 10-11) and in particular for treating cirrhosis (claims 8-9).

The application US2016/0051501 relates to a method for treating a viral or alcohol-related or immune liver illness by a compound of formula.

Elafibranor is not cited in other patents. Only results of studies appear in several articles, the first of which were published in 2007 (Fruchart, Am J Cardiol 2007; 100[suppl]:41N-46N; in 2013: Fruchart Cardiovascular Diabetology 2013, 12:82).

The posting “The hepatic and extra-hepatic profile of resolution of steatohepatitis induced by GFT-505 (elafibranor)” by Sanyal A J et al., deals with the results of a phase 2b study (Golden505) proposing a daily dose of 80 or 120 mg of elafibranor administered to 270 NASH patients (3 groups including diabetics and non-diabetics). There is no information about the pharmaceutical composition of the capsules dosed at 40 mg used for this study or on the physical and chemical characteristics of the elafibranor or the rationale concerning the administration before breakfast.

The pharmacokinetic parameters including metabolism are not publically available for elafibranor despite the phase 1 studies that have been carried out. In 2012, in the dose research study “Comparative Bioavailability—Gender Effect—Single and Multiple Ascending Dose Safety and Pharmacokinetic Study of GFT505”, changes were made by Genfit in the formulations of elafibranor. A study of the relative bioavailability between new and old formulations was carried out, on a dose range of up to 300 mg. There is no publication of results or information that justify and support the reasons for this formulation work.

Metformin, the chemical name of which is 3-(diaminomethylidene)-1,1-dimethylguanidine, has a structure according to FIG. 3 (formula I), and with the chemical formula C₄H₁₁N₅. The substance is known as an active principle in a drug belonging to the antidiabetic biguanides class having an antihyperglycaemic action (Glucophage®, Glumetza®, etc). Metformin is associated with a very low incidence of lactic acid. It helps to reduce levels of LDL cholesterol and triglycerides, and is not associated with weight gain, and prevents the cardiovascular complications of diabetes. Metformin is not metabolised and is excreted unchanged by the kidneys.

The molecular weight of the active molecule is 129.16 g/mol and has a melting point of 223-226° C. The hydrochloride salt of metformin is most used as an active principle in drugs present on the market such as Glucophage®, because of very good solubility in water (Log P−0.5 and pKa 12.4) and established chemical stability even under high temperature and humidity conditions (40° C./75% RH to ICH standard).

The applicant has wished to improve the efficacy of elafibranor.

Surprisingly, it found that elafibranor in salt form with metformin has advantageous effects different from the sum of elafibranor and metformin taken individually. This is because the metformin salt affords a synergy of action of the active principles in particular influencing the bioavailability thereof.

### BRIEF DESCRIPTION OF THE FIGURES

The aims, objects, features and advantages of the invention will emerge more clearly from the detailed description of an embodiment thereof that is illustrated by the following accompanying figures, in which:

FIG. 1: chemical formula of elafibranor;

FIG. 2: common chemical grouping of fibrates and elafibranor;

FIG. 3: chemical formula of metformin;

FIG. 4: derived chemical formula of substituted 1,3-diphenylprop-2-en-1-one comprising elafibranor.

FIG. 5: General formula of a compound of the patent application EP2504005 comprising elafibranor.

FIG. 6: synthesis diagram of elafibranor (GFT505)

FIG. 7A: 1H NMR spectrum elafibranor (GFT505)

FIG. 7B: UPLC MS elafibranor (GFT505)

FIG. 7C: UV spectrum of elafibranor (GFT505)

FIG. 8A: 1H MNR spectrum elafibranor (GFT505) metformin salt

FIG. 8B: UPLC MS/UV spectrum elafibranor (GFT505) metformin salt

FIG. 9: UPLC MS elafibranor (GFT505) metformin salt powder after 14 days in powder form (group 1 light, group 2 light protection)

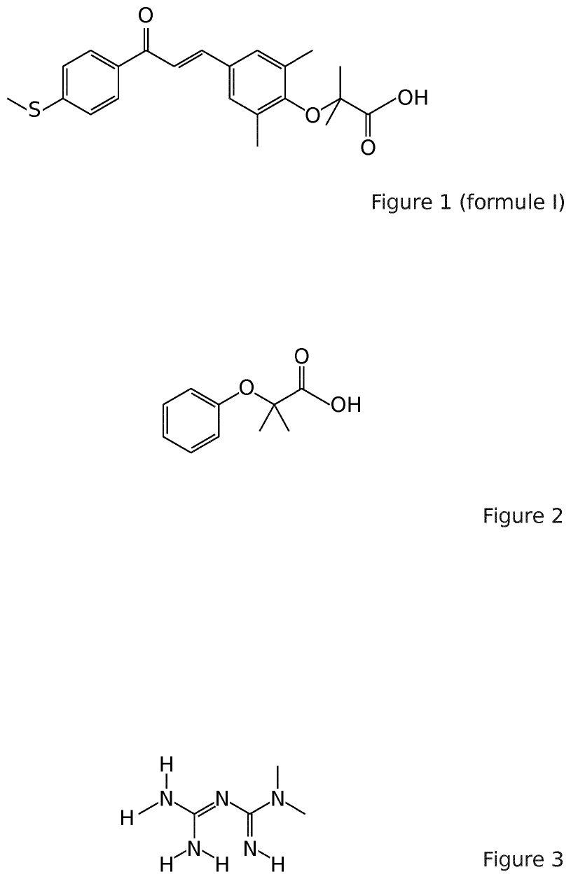

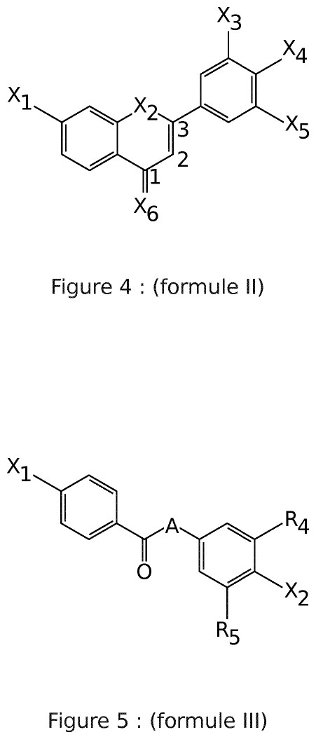

### DISCLOSURE OF THE INVENTION

Before beginning a detailed review of embodiments of the invention, optional features, which can optionally be used in association or alternatively, are set out below.

It is stated first of all that the invention relates to a composition comprising, as active principle, a pharmaceutically acceptable elafibranor (GFT505) metformin salt.

Advantageously, the invention relates to a composition comprising at least one active principle, characterised in that the at least one active principle comprises an elafibranor metformin salt.

Advantageously, the composition is intended to treat and/or prevent illnesses resulting from the metabolic syndrome comprising diabetes, obesity, liver and cardiovascular diseases and dyslipidaemia.

Advantageously, the composition is intended to treat and/or prevent liver diseases chosen from non-alcoholic hepatic steatoses, non-alcoholic steatohepatitis, fibrosis, cirrhosis and cancers.

Advantageously, the composition is intended to treat and/or prevent liver diseases, characterised in that the liver disease consists of non-alcoholic hepatic steatosis (NAFLD).

Advantageously, the composition is intended to treat or prevent liver diseases, characterised in that the liver disease consists of non-alcoholic steatohepatitis (NASH).

Advantageously the composition is intended to treat or prevent obesity.

Advantageously the composition is in a form suitable for oral administration.

Advantageously, the composition is in a form suitable for parenteral administration.

Advantageously the composition comprises at the very most 500 mg of elafibranor metformin salt.

Oral administration methods afford simple and rapid take-up of the pharmaceutical composition.

Advantageously, the composition is in a form suitable for intravenous administration.

Advantageously the composition is in a form suitable for subcutaneous administration.

Advantageously the composition comprises at least one excipient chosen from binders, disintegrating agents, diluents, lubricants, surfactants, buffers, flow agents, dyes, flavourings, sweeteners, solvents or preservatives.

The invention also relates to a pharmaceutically acceptable elafibranor metformin salt in accordance with the formula: C₂₂H₂₃O₄S.C₄H₁₁N₅.

Advantageously, the drug form of the composition consists of a powder for an injectable solution.

Advantageously, the drug form consists of a powder for oral suspension.

The pharmaceutically acceptable salt of elafibranor has the advantage of having better solubility in water compared with the basic form.

Advantageously, the drug form consists of the form of an injectable solution, a tablet, a dispersible tablet, an orodispersible tablet, a capsule, a soluble tablet, a freeze-dried product, an effervescent tablet, a tablet to be chewed, a prolonged-release tablet or a sachet.

Advantageously, the profile of dissolution in an acid environment, in water and in FaSSIF and FeSSIF medium simulating the taking of meals, the pharmaceutically acceptable elafibranor metformin salt has a dissolution percentage greater than 90% after 30 minutes.

The invention relates to a use of a composition comprising, as active principle, a pharmaceutically acceptable elafibranor (GFT505) metformin salt for obtaining a drug intended for use in the treatment or prevention of illnesses resulting from the metabolic syndrome, with in particular a dual action for treating obesity associated with non-alcoholic steatohepatitis (NASH) and hypertriglyceridaemia.

In another aspect, the invention relates to the preparation of the pharmaceutically acceptable elafibranor (GFT505) metformin salt demonstrating physical and chemical properties that are more advantageous than the free basic form of elafibranor, in particular with regard to solubility and/or stability.

### DETAILED DESCRIPTION

The present invention relates to the use of a pharmaceutically acceptable elafibranor metformin salt in one of its crystalline forms, optionally polymorphous, or amorphous, in the preparation of a drug for treating or preventing illnesses, in particular illnesses relating to the metabolic syndrome and having a dual action with regard to obesity and hepatic steatoses.

The invention also relates to the use of a pharmaceutically acceptable salt of metformin of 2-[2,6 dimethyl-4-[3-[4-(methylthio)phenyl]-3-oxo-1(E)-propenyl]phenoxyl]-2-methylpropanoic acid, of chemical formula C₂₆H₃₄O₄N₅S, able to be used in a pharmaceutical composition for preventing or treating illnesses, in particular illnesses resulting from the metabolic syndrome such as obesity, insulin resistance and liver diseases including non-alcoholic steatohepatitis NASH.

The elafibranor metformin salt is also referred to as elafibranorate metformin salt.

The pharmaceutical composition of the invention can be administered enterally, parenterally, topically or subcutaneously. According to one administration mode, the composition is administered enterally, such as, for example, a tablet, a capsule, a soft capsule, a freeze-dried product, a dispersible, orodispersible, effervescent or soluble tablet, an oral solution or a powder for oral suspension.

According to a preferred administration mode, the composition is administered orally in the form of tablets or capsules.

According to a preferred administration mode, the composition is administered intravenously or subcutaneously, in the form for example of an injectable solution or a powder for an injectable solution.

The formulations intended to be administered intravenously or orally contain an elafibranor metformin salt that is crystallised or has an amorphous structure in order to optimise the method for manufacturing the speciality where applicable.

One of the pharmaceutically preferred compositions of the invention is a powder for oral suspension or for an injectable preparation that is soluble and stable under normal conditions of temperature and humidity.

The present invention therefore relates, as a novel product, to the salt of metformin and of 2-[2,6 dimethyl-4-[3-[4-(methylthio)phenyl]-3-oxo-1(E)-propenyl]phenoxyl]-2-methylpropanoic acid, and also to the preparation of the salt of metformin and of this 2-[2,6dimethyl-4-[3-[4-(methylthio)phenyl]-3-oxo-1(E)-propenyl]phenoxyl]-2-methylpropanoic acid.

This preparation can be carried out by a method of salification of 2-[2,6 dimethyl-4-[3-[4-(methylthio)phenyl]-3-oxo-1(E)-propenyl]phenoxyl]-2-methylpropanoic acid by dimethylbiguanide. The preparation method is given in the following examples.

### EXAMPLES

### Example 1

### Synthesis and Characterisation of Elafibranor (GFT505)

The applicant decided to prepare samples of elafibranor for assessing the feasibility of the steps of synthesising this molecule and characterising the physical and chemical properties of the product obtained. The operating method is derived from the information described in the patent EP 1525177 B1 for synthesising compound 29. The steps are reproduced identically.

Experimental Protocol

The compound is synthesised from 1-[4-methylthiophenyl]-3-[3,5-dimethyl-tertiobutyloxycarbonyldimethylmethyloxyphenyl]prop-2-en-1-one.

### Step 1: 1-[4-methylthiophenyl]-(E)-3-[3,5-dimethyl-4-hydroxyphenyl]prop-2-en-1-one (Intermediate 1)

4-methylacetophenone (20 g, 0.12 mol, 1 eq) and 3,5-dimethyl-4-hydroxybenzaldehyde (18 g, 0.12 mol, 1 eq) are solubilised in 300 ml of 4N HCl in dioxane. The reaction medium is stirred for 30 hours and then the solvents are evaporated. Purification by hot recrystallisation in 70 ml of isopropanol and 12 ml of water: 30 g (yellow solid, yield: 92%).

Raw formula: C₁₈H₁₈O₂S

ESI-MS m/z=299.18 [M+H]+

1H NMR DMSO-d6 δ ppm: 2.18 (s, 6H), 2.53 (s, 3H), 7.36 (d, J=8.5 Hz, 2H), 7.47 (s, 2H), 7.57 (d, J=15.5 Hz, 1H), 7.69 (d, J=15.5 Hz, 1H), 8.05 (d, J=8.5 Hz, 2H), 8.93 (s, 1 H)

### Step 2: 1-[4-methylthiophenyl]-(E)-3-[3,5-dimethyl-4-tertiobutylcarbonyldimethylmethyloxyhenyl]prop-2-en-1-one (Intermediate 2)

Caesium carbonate (87 g, 0.134 mol, 4 eq) and tetrabutylammonium iodide (12 g, 0.033 mol, 0.5 eq) are added to an intermediate solution 1 (20 g, 0.067 mol, 1 eq) in 50 ml of a DMSO/water mixture (3/2). The reaction medium is stirred for 30 minutes at 80° C. and tert-butyl bromoisobutyrate (30 g, 0.134 mol, 2 eq) is added. Then 2 additions of 2 eq of tert-butyl bromoisobutyrate diluted to 50% in DMSO are each made at an interval of 1 hour. The reaction medium is stirred for 2 days at 80° C. The reaction medium is left to cool to ambient temperature and then 1.5 litres of water are added and the product is extracted with dichloromethane (4 times). The organic phase is dried on a phase-separation cartridge and evaporated dry. Purification on silica gel (cyclohexane/ethylacetate: 95/5 to 80/20): 18 g (orange solid, yield: 61%).

Raw formula: C₂₆H₃₂O₄S

ESI-MS m/z=441.33 [M+H]+

1H NMR DMSO-d6 δ ppm: 1.36 (s, 6H), 2.19 (s, 6H), 2.48 (broad peak, H2O+9H), 2.54 (s, 3H), 7.38 (d, J=8.4 Hz, 2H), 7.55 (s, 2H), 7.59 (d, J=15.6 Hz, 1 H), 7.80 (d, J=15.6 Hz, 1H), 8.07 (d, J=8.4 Hz, 2H)

### Step 3: 1-[4-methylthiophenyl]-(E)-3-[3,5-dimethyl-4-carboxydimethylmethyloxyhenyl]prop-2-en-1-one

Intermediate 2 (25 g, 0.057 mol, 1 eq) is solubilised in 50 ml of dichloromethane, and 22 ml of trifluoroacetic acid (5 eq, 0.284 mol) is added gently. The reaction medium is stirred for 3.5 hours at ambient temperature and then the solvents are evaporated dry. Purification on silica gel (dichloromethane/methanol: 100/0→95/5); 13 g (yellow solid, yield 60%).

Raw formula: C₂₂H₂₄O₄S

ESI-MS m/z=385.25 [M+H]+

1H NMR DMSO-d6 δ ppm: 1.36 (s, 6H), 2.19 (s, 6H), 2.54 (s, 3H), 7.37 (d, J=8.6 Hz, 2H), 7.55 (s, 2H), 7.59 (d, J=15.6 Hz, 1 H), 7.80 (d, J=15.6 Hz, 1 H), 8.07 (d, J=8.6 Hz, 2H), 12.94 (s, 1H)

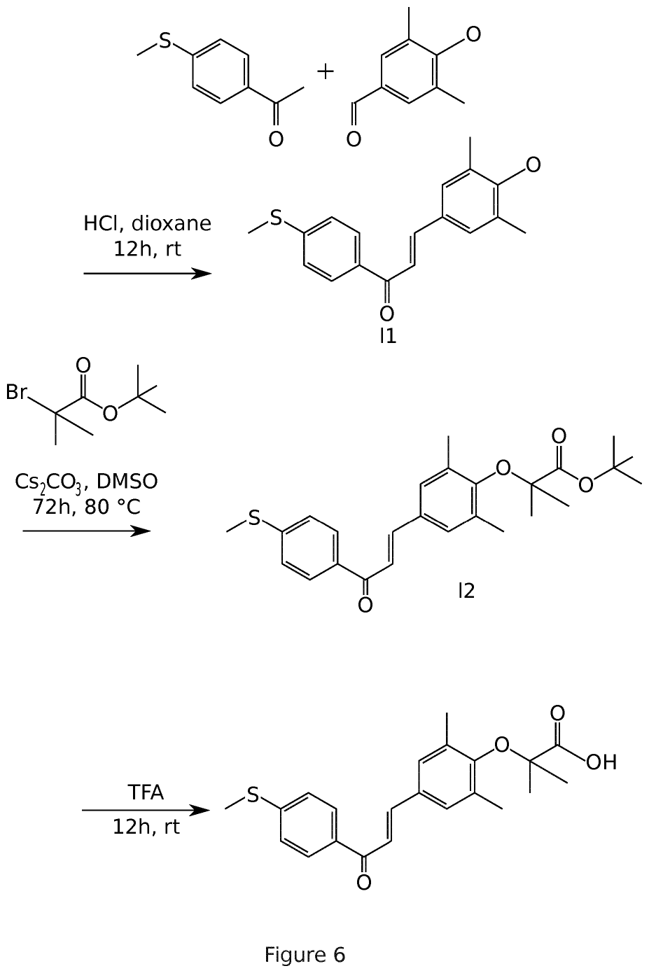

The reaction diagram is given in FIG. 6.

Results

The analysis data of the 10.1 g of the batch obtained (EM0274L2) are summarised below:

Molecular mass: 384.5 (exact mass: 384.1);

1H NMR spectrum: conforming to the structure, cf spectrum in FIG. 6A below.

LCMS: TR=1.42 mn, m/z: 385.00=[M+H]+;

Purity: >98% (1H NMR and LCMS);

Melting point: 144-145° C.

The appearance of the product is an amorphous yellow solid powder. The product exhibits significant absorption in the near visible with an apex at approximately 347 nm.

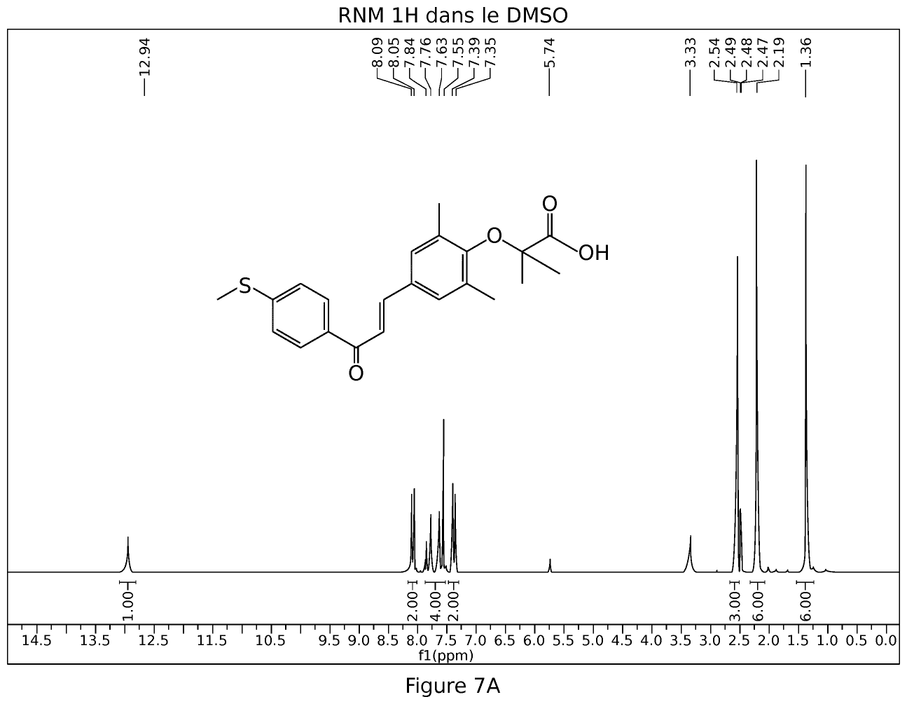

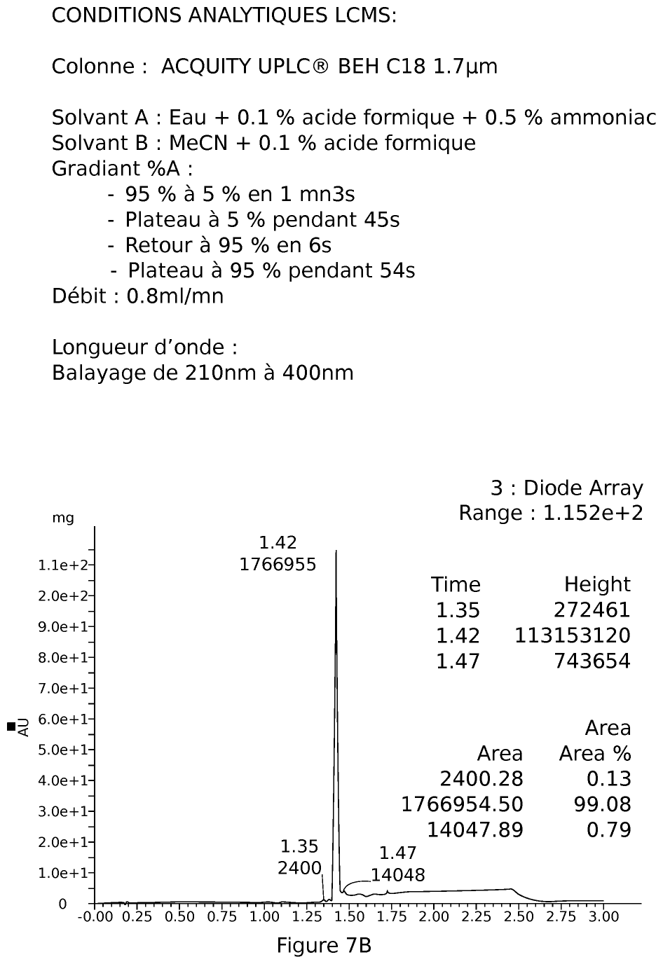

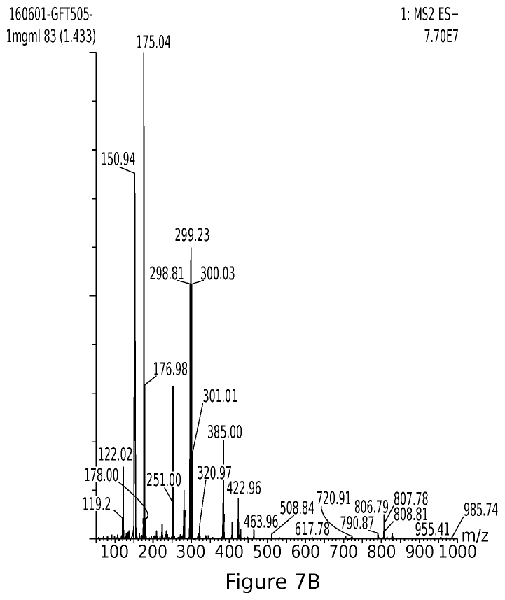

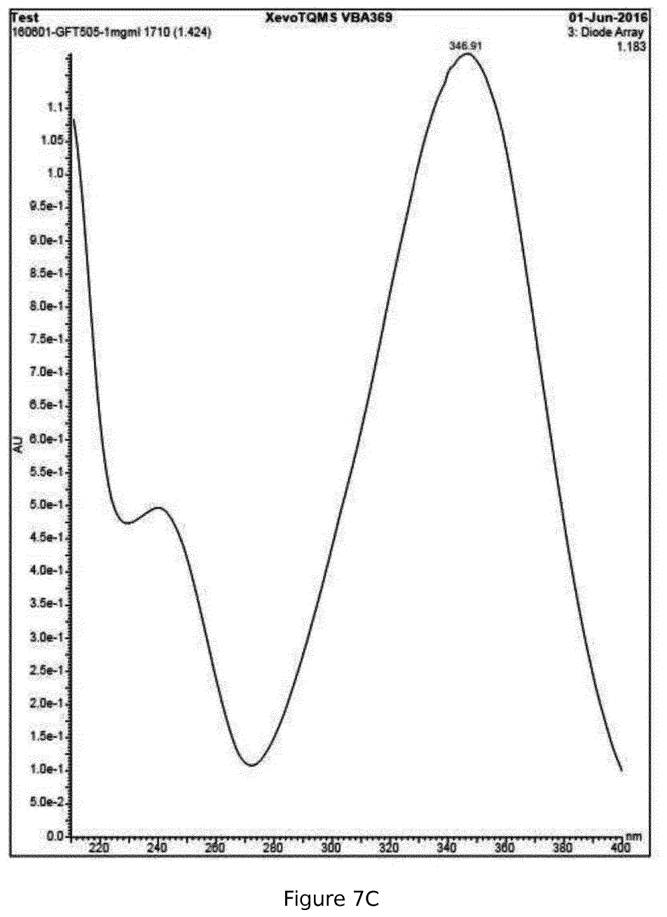

The information is detailed in the following FIG. 7.

The product obtained is conforming in terms of chemical purity and demonstrates absorption in the near visible that requires that the chemical stability under light and phototoxicity must therefore be checked.

### Example 2

### Measurement of Solubility of Elafibranor (GFT505)

During an experiment carried out for the invention, it was shown that elafibranor (GFT505) had a chemical structure similar to the fibrate family (FIG. 2). Elafibranor being a carboxylic acid, the applicant chose to check the solubility in water of this molecule in order to rule on the feasibility of development of pharmaceutical compositions in accord with the expectations of patients, and that were more effective and tolerated by the patients.

Experimental Protocol

The thermodynamic solubility of the basic elafibranor is studied over a period of 24 hours and 72 hours in various aqueous buffers in the presence or not of surfactants. Batch number EM0274L2 is used for this work. The product is dissolved in the solvents indicated in table 1. After 24 hours and 72 hours of incubation at ambient temperature (22°-24° C.), the solutions are sampled and then filtered over 0.2 μm polycarbonate filters in flasks for LCMS analyses, and diluting once in DMSO before stirring for 2 minutes (vortex or sonification).

Results

The thermodynamic solubility results are given in table 1 below:

TABLE 1
Thermodynamic solubility of elafibranor in aqueous medium

| | |
|---|---|
| Solubility | Solubility |
| after 24 hours | after 72 hours |

| Solvents | μM | mg/ml | μM | mg/ml |
|---|---|---|---|---|
| Buffer pH 4.6 | 114 | 0.044 | 110 | 0.042 |
| Buffer pH 7.4 | 504 | 0.194 | 609 | 0.234 |
| Buffer pH 8.5 | 4419 | 1.701 | 4270 | 1.644 |
| Propylene glycol | 33520 | 12.905 | 35081 | 13.506 |
| Polyethylene | 39022 | 15.023 | 40971 | 15.774 |
| glycol 400 | | | | |

The solubility of elafibranor is low in an aqueous medium. Increases as a function of the pH, changing from 114 to 4419 μm and from pH 4.6 to 8.5. The addition of cosolvent such as propylene glycol or PEG 400 significantly improves the solubility of the molecule.

### Example 3

### Preparation of Elafibranor (GFT505) Metformin Salts

The applicant prepared elafibranor metformin salt for the purpose of determining its characteristics and to compare them with elafibranor in free basic form, with the objective of producing pharmaceutical compositions.

Experimental Protocol

The batches of elafibranor metformin salts are produced from a batch of free basic elafibranor.

1 g of GFT505 (2.6 mmol) and 104 mg of NaOH (1 eq, 2.6 mmol) are put in suspension in 8 ml of isopropanol and 10 ml of methanol and heated to 65° C. A solution of 431 mg of metformin.Cl (1 eq, 2.6 mmol) in 2 ml of isopropanol is added to the yellow reaction medium and stirred for 30 minutes at 65° C. After returning to ambient temperature, the fine suspension is filtered quickly, washed by 2×1 ml of isopropanol. After approximately 30 minutes, a pale yellow solid begins to precipitate. After 24 hours at ambient temperature, the solid is filtered, washed with 2×1 ml of isopropanol and dried for 24 hours at 45° C. under vacuum. The yellow residue is resolubilised in 10 ml of isopropanol and 6 ml of methanol at 65° C. Heating is stopped and the clear reaction medium is left to cool under stirring. A solid begins to precipitate at around 47° C. After 24 hours, the solid is filtered, washed with 2×1 ml of isopropanol and dried for 72 hours at 45° C. under vacuum (batch CP0685L1, see Report No. 1 of 30 August 2016). After 10 days of additional drying at 45° C. under high vacuum (<10-2 mbar), the traces of solvent detected in the previous batch have disappeared (batch CP0685L2).

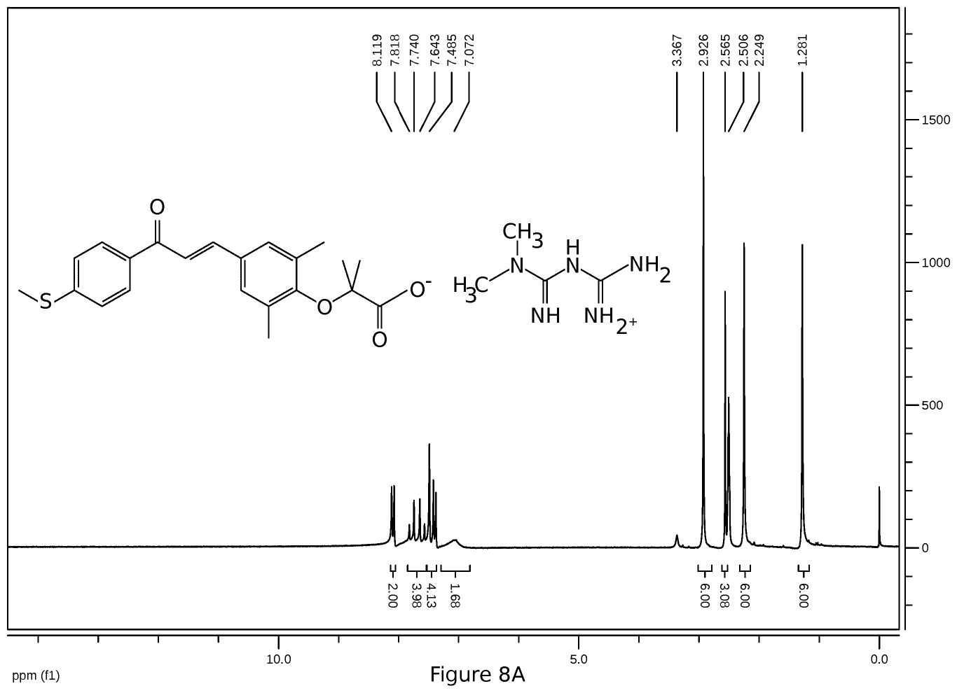

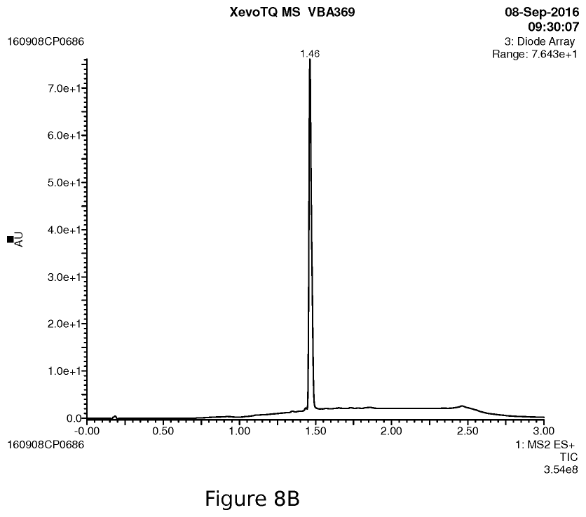

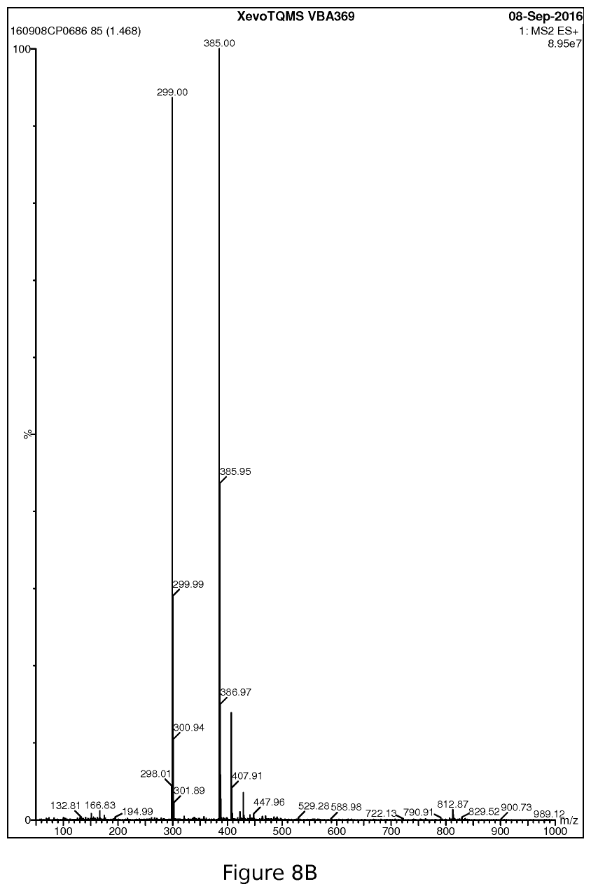

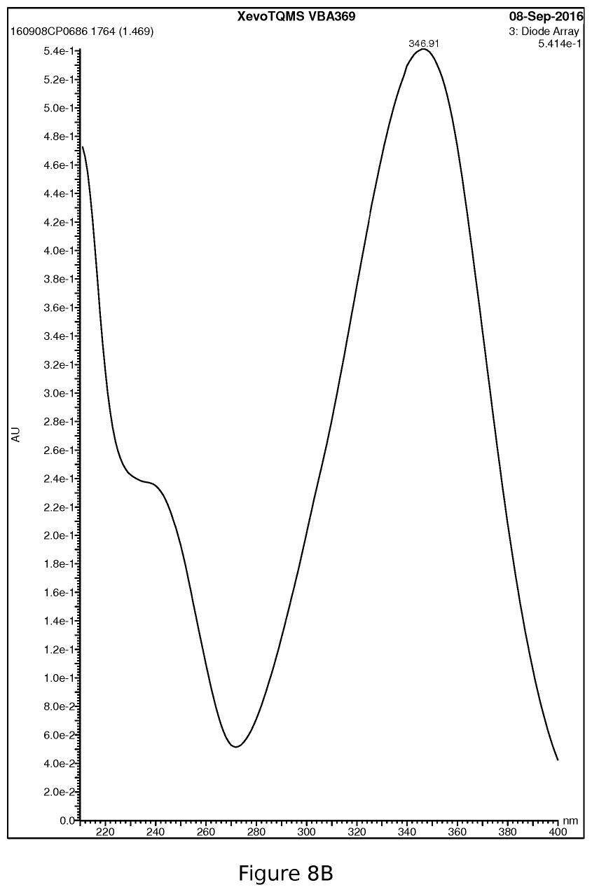

The products are next stored cool (2°-8° C.) under inert gas to prevent any degradation. An analysis of the product is carried out, including identification and chemical purity (see FIG. 8).

Results:

The results were as follows:

Molecular mass: 513

Appearance: amorphous yellow solid.

Melting point: 178/180° C.

TR=1.46 mn, m/z: 385=[M+H]+

Structure:

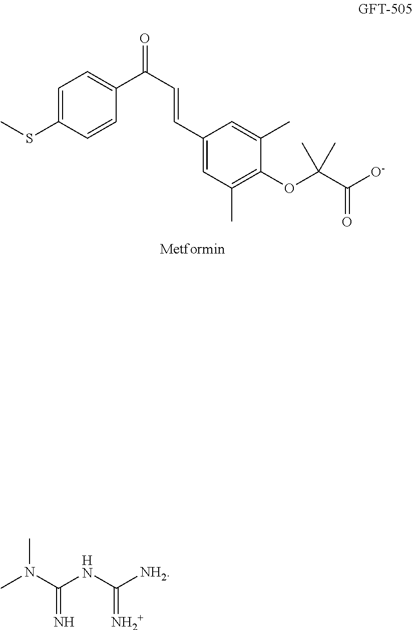

Other data illustrated in FIG. 8.

### Example 4

### Solubility Test on the Elafibranor (GFT505) Metformin Salt

This example presents the solubility characteristics of various forms and elafibranor salts, with a view to parenteral administration or in the context of a fast-release enteral composition.

The solubility kinetics is determined in an aqueous medium (water and pharmacopoeia buffers), at ambient temperature. The summarised results are indicated in the following table.

TABLE 2
Solubility of the elafibranor metformin salt

| | |
|---|---|
| | Elafibranor |
| Elafibranor | (GFT505) |
| (GFT505) | Amorphous |
| metformin salt | free basic |

| Solvents | μM | mg/ml | μM | mg/ml |
|---|---|---|---|---|
| Buffer pH 4.6 | ND | ND | 114 | 0.044 |
| Buffer pH 7.4 | 12000 | 6 | 504 | 0.194 |
| Buffer pH 8.5 | ND | ND | 4419 | 1.701 |
| Propylene glycol | — | — | 33520 | 12.905 |
| Polyethylene glycol | — | — | 39022 | 15.023 |
| 400 | | | | |

The elafibranor metformin salt is approximately 20 times more soluble than elafibranor in its free basic form.

### Example 5

### Stability of Elafibranor and One of the Salts Thereof

The applicant decided to check the stability of elafibranor and the metformin salts thereof after exposure to light and temperature.

Experimental Protocol

Samples were prepared in the form of powder alone and aqueous solutions for the following samples: elafibranor, elafibranor metformin.

The stability is measured over a period of 7 to 14 days by UPLC MS, with calculation of the degree of recovery of the elafibranor peak with respect to the initial value and measurement of its purity index. The products were exposed to daylight and to ambient temperature.

The reference samples for their part are stored cool (2°-8° C.), protected from light by aluminum paper and under inert gas for the solid product.

Results

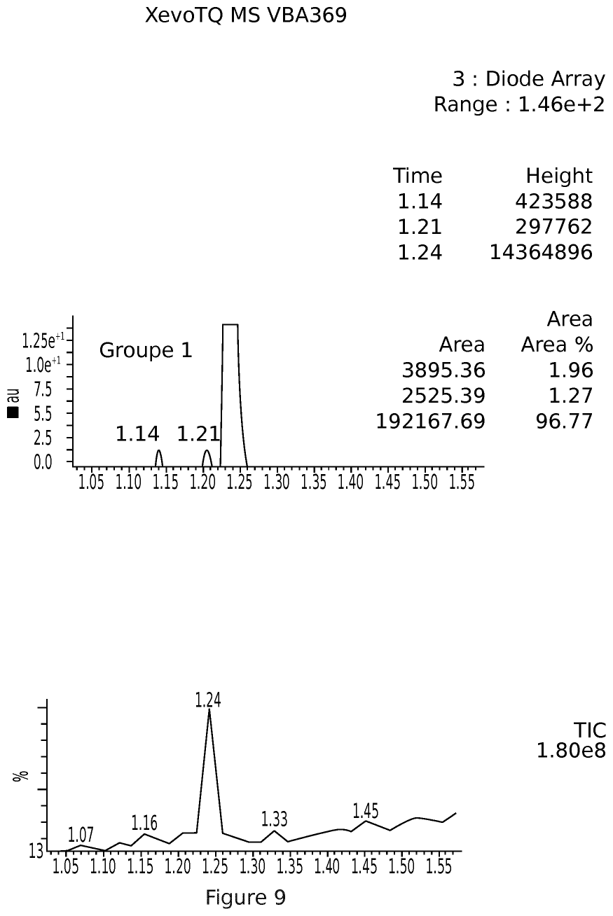

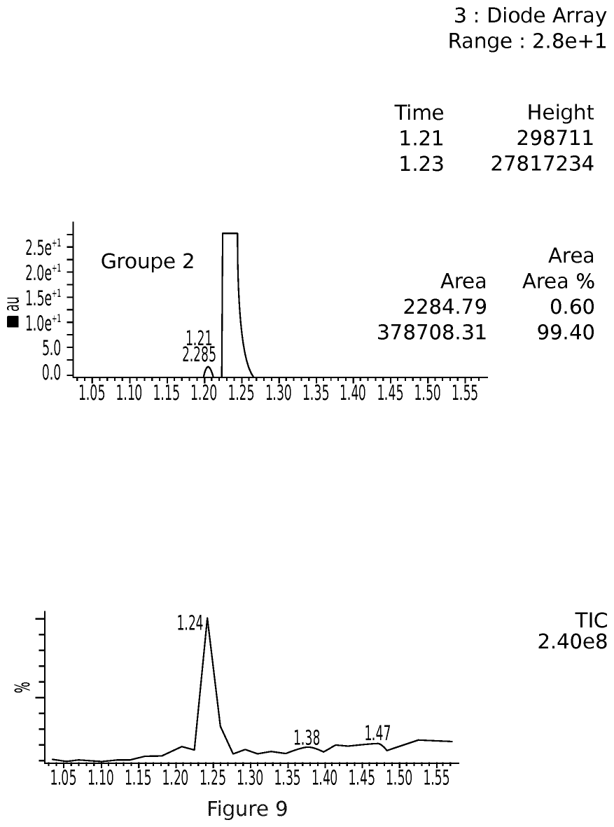

The results demonstrate that the free basic elafibranor is more sensitive to light (photosensitivity), whether in powder form or in solution compared with the salt. The elafibranor metformin salt has an appreciable change in colouring of the yellow powder into darker yellow following the same tendency as the product in its free basic form. Nevertheless, the recovery after 14 days of storage lies within the norm 100±5%: detection of degradation products (<5%). FIG. 9 for the LCMS (group 1 with light, group 2 with protection via aluminum for the powder samples).

The temperature has no impact on stability. The products of degradation under light were not identified.

1. A composition comprising at least one active principle, wherein the at least one active principle comprises an elafibranor metformin salt.

2. The composition according to claim 1 for use thereof for treating or preventing illnesses resulting from the metabolic syndrome comprising diabetes, obesity, liver and cardiovascular diseases and dyslipidaemia.

3. The composition according to claim 1 for use thereof for treating or preventing liver diseases chosen from non-alcoholic hepatic steatoses, non-alcoholic steatohepatites, fibroses, cirrhoses and cancers.

4. The composition according to claim 3 for use thereof for treating or preventing liver diseases, wherein the liver disease consists of non-alcoholic hepatic steatosis (NAFLD).

5. The composition according to claim 3 for use thereof for treating or preventing liver diseases, wherein the liver disease consists of non-alcoholic steatohepatitis (NASH).

6. The composition according to claim 2 for use thereof for treating or preventing obesity.

7. The composition according to claim 1, wherein the composition is in a form suitable for oral administration.

8. The composition according to claim 1, wherein the composition is in a form suitable for parenteral administration.

9. The composition according to claim 1, wherein the composition is in a form suitable for intravenous administration.

10. The composition according to claim 1, wherein the composition is in a form suitable for subcutaneous administration.

11. The composition according to claim 1, comprising at least one excipient chosen from binders, disintegrating agents, diluents, lubricants, surfactants, buffers, flow agents, dyes, flavourings, sweeteners, solvents or preservatives.

12. The pharmaceutically acceptable elafibranor metformin salt in accordance with the formula C₂₂H₂₃O₄S.C₄H₁₁N₅: [chemical structure]
